(19) United States
(12) Patent Application Publication
(10) Pub. No.: US 2014/0364321 A1
(43) Pub. Date: Dec. 11, 2014

(54) Method for analyzing DNA methylation based on MspJI cleavage

(71) Applicant: BGI TECH SOLUTIONS CO., LTD., Shenzhen, Guangdong (CN)

(72) Inventors: Hanlin Lu, Shenzhen (CN); Jun Wang, Shenzhen (CN); Jian Wang, Shenzhen (CN); Huanming Yang, Shenzhen (CN)

(21) Appl. No.: 14/369,447

(22) PCT Filed: Dec. 31, 2011
(86) PCT No.: PCT/CN2011/002242

§ 371 Date: Jun. 27, 2014

(51) Int. Cl. C12Q 1/68; G06F 19/18; G06F 19/22

(52) U.S. Cl. CPC C12Q 1/6809; G06F 19/22; G06F 19/18; C12Q 1/6869
USPC 506/2; 702/20

(57) ABSTRACT

Provided is a method for detecting DNA methylation based on MspJI cleavage and performing bioinformatics analysis of genomic methylation.

### CROSS-REFERENCE TO RELATED APPLICATION

This Application is a Section 371 National Stage Application of International Application No. PCT/CN2011/002242, filed Dec. 31, 2011 and published as WO/2013/097060 A1 on Jul. 4, 2013, in English, the contents of which are hereby incorporated by reference in their entirety.

### TECHNICAL FIELD

Embodiments of the present disclosure generally relate to a field of bioinformatics, more particularly, to an effective and accurate bioinformatics analysis method for study plant genome methylation.

### BACKGROUND

Modification of DNA methylation is one important aspect in epigenetics research, serving in many biological phenomenon and processes, for example: dosage compensation, DNA site polymorphism, transposon silence and etc. Current methods of studying DNA methylation combined with high-throughput sequencing technology comprise: bisulfite sequencing (BS-sequencing), methyl-binding protein (MBD) by means of methylated-cytosine combining protein, methylated DNA immune-precipitation (MeDIP) by means of antibody capture site, reduced representation bisulfite sequencing (RRBS) by means of methylated-cytosine site-specific enzyme digestion, and etc. MBD sequencing is more sensitive to parts with a hypermethylation and a medium density of CpG, MeDIP-sequencing is more sensitive to parts with a hypermethylation and a high density of CpG, however, both are not accurate enough. Although the BS-sequencing can accurately analyze a methylation status of each C base and plot a DNA methylation map in a single-base resolution, it requires large volume of sequencing data with a high cost of sequencing. The reduced representation bisulfite sequencing (RRBS) is based on bisulfite sequencing (BS), comprising: firstly selecting a partial region in a whole genome by an enzyme digestion technology, and then performing BS-sequencing, which has some advantages in cost comparing with BS-sequencing, however, it has difficulties in enriching large amount of mCHG and mCHH in a methylation form from a plant sample.

Therefore, currently an effective and accurate method for study plant genome methylation still needs to be developed.

### SUMMARY

In order to realize a detection of DNA methylation by a massive sequencing without BS sequencing, the present disclosure provides a bioinformatics analyzing method for detecting a DNA methylation based on MspJI digestion, in which MspJI is a modification-dependent restriction enzyme. A method of enriching a methylated site by MspJI digestion does not need to subject a whole genome to a bisulfite treatment, which only obtains information of the methylated site and nearby sequence thereof Such method yields a lower data volume in relative to a whole genome bisulfite sequencing, which is a simple and convenient methylation sequencing method with a moderate operating condition. Accordingly, a bioinformatics analyzing method correspondingly is designed, to determine a recognition site, a methylation site and a type thereof in an enzyme-digested fragment, and embodiments of the subsequent analyzing method are also provided.

In one aspect, there is provided a method of detecting a genome DNA methylation, comprising following steps:
1) digesting a genome DNA sample with MspJI, to obtain fragments,
2) sequencing the fragments, to obtain reads;
3) aligning the reads to a reference sequence, to select a uniquely aligned read; and
4) determining a site in the reference sequence being methylated in the uniquely aligned read;
wherein the site in the reference sequence corresponds to a C site in at least one of YNCGNR, YCNGR, CLANG, GNNC, CYNRG, CNYRNG, YNNGCNNR, YNNGNCNNR, CNNR, YNNG, and a complementary strand thereof.

In another aspect, there is provided a method of analyzing a genome methylation, comprising following steps:
1) digesting a genome DNA sample with MspJI, to obtain fragments,
2) sequencing the fragments, to obtain reads;
3) aligning the reads to a reference sequence, to select a uniquely aligned read;
4) determining a methylated C site in the uniquely aligned read, to determine a corresponding site in the reference sequence being methylated,
wherein the methylated C site is a C site in at least one of YNCGNR, YCNGR, CNNG, GNNC, CYNRG, CNYRNG, YNNGCNNR, YNNGNCNNR, CNNR, YNNG; and a complementary strand thereof;
5) calculating a type distribution of CG, CHG or CHH in the methylated C site, wherein H is C, A or T;
6) annotating following one or more kinds of information in a whole genome map, to obtain a whole genome methylation map, comprising:
a sequencing depth of each methylated C site;
information, comprising methylated single nucleotide annotation, No. of chromosome in which each determined methylated C site locates, a C site position, forward or reverse strand, a coverage depth, a digested and recognized site, types of cytosine; and
a total amount and coverage of the methylated cytosine position.

Further detailed description will be given combining with following Figures and embodiments to make the purpose, technical solution and advantages more obvious and clear. It should understand that specific examples described herein are used for explaining but not limiting the present disclosure.

### BRIEF DESCRIPTION OF THE DRAWINGS

FIG. 1 is a flow chart showing specific examples of the present disclosure.

FIG. 2 is a schematic diagram showing a recognition site obtained by restriction enzyme MspJI in the present disclosure. MspJI recognizes methylated double-strand site in the context of CNNR(R=A or G), and introduces double-stranded breaks at fixed distances of 9 by and 13 by on R end, leaving a four-base 5′ overhang. If a recognition site is fully methylated, i.e., all corresponding sites in the double-strand exist methylated MspJI-recognized site, then an enzyme-digested fragment having a length of 30 to 32 bases is yielded by two-way cleavage, which is the emphasis in the present disclosure.

FIG. 3 is a detection result of genome integrity in Arabidopsis sample, showing Arabidopsis genome quality for enzyme-digestion by 1% agarose gel electrophoresis detection. It can be seen that the genome integrity of Arabidopsis is excellent, without contamination and degradation, which may be used for subsequent enzyme digestion reaction.

FIG. 4 is a result of fragments obtained by MspJI-digested Arabidopsis genome having a length of 26 by to 38 by and recycled by 15% native polyacrylamide gel, the left panel shows the 15% native polyacrylamide gel prior a fragment selection, the right panel shows the 15% native polyacrylamide gel after the fragment selection. By comparison, it can be seen that enriched short fragments in an appropriate range of approximately 30 by are recycled, which can be used for subsequent library construction.

FIG. 5 is a result of target fragments obtained by PCR amplification and recycled by 2% agarose gel electrophoresis, the left panel shows the 2% agarose gel prior to library recycling, the right panel shows the 2% agarose gel after library recycling. Approximately 150 by is the fragment size of the target fragments after ligating to an adaptor and extended by PCR amplification, and fragments in a range of 146 by to 158 by here are recycled which may accurately select the original target fragments having a length of 26 by to 38 bp. Thus the constructed library may be used for investigating most fully methylated recognition sites digested by MspJI, i.e., symmetry methylated CpG, CHG, CHH site.

FIG. 6 is a schematic diagram showing every type of enzyme-digested site in Arabidopsis genome (upper left), a type of methylated cytosine (upper right), and sequence logo of YNCGNR site (bottom). It can be seen from FIG. 6 that, except a type of one-way enzyme-digested site, an overwhelming majority of the enzyme-digested fragments in the two-way enzyme-digested fragments are YNCGNR, YCNGR and CNNG; the sequence Logo in the bottom panel reflects a distribution of base conservatism in sequences containing YNCGNR site.

FIG. 7 is a schematic diagram showing a distribution trend of a methylated cytosine site which is determined in Chromosome 1 of Arabidopsis.

FIG. 8 shows Arabidopsis genes and a distribution of a methylated cytosine site upstream and downstream thereof (upper left), a statistical distribution of a methylated cytosine in a repetitive sequence region (upper right), as well as a schematic diagram illustrating a methylated cytosine, a repetitive sequence, and a distribution of reads coverage within every window of a whole genome (bottom).

FIG. 9 is a schematic diagram showing a correlation between Arabidopsis whole genome methylation data obtained by a method of detecting enzyme-digested methylation and BS sequencing data.

### DETAILED DESCRIPTION

In DNA sequence of the present disclosure,
Y represents C or T;
R represents A or G;
N represents A, C, T or G;
H represents C, A or T.

In the present disclosure, reads refer to sequencing fragments output from sequencer and prior to connecting.

A restriction endonuclease MspJI being sensitive to methylation and having a more divergent homology to E. coli Mrr is used in the present disclosure, which is commercially available, for example, being obtained from New England Biolabs (NEB).

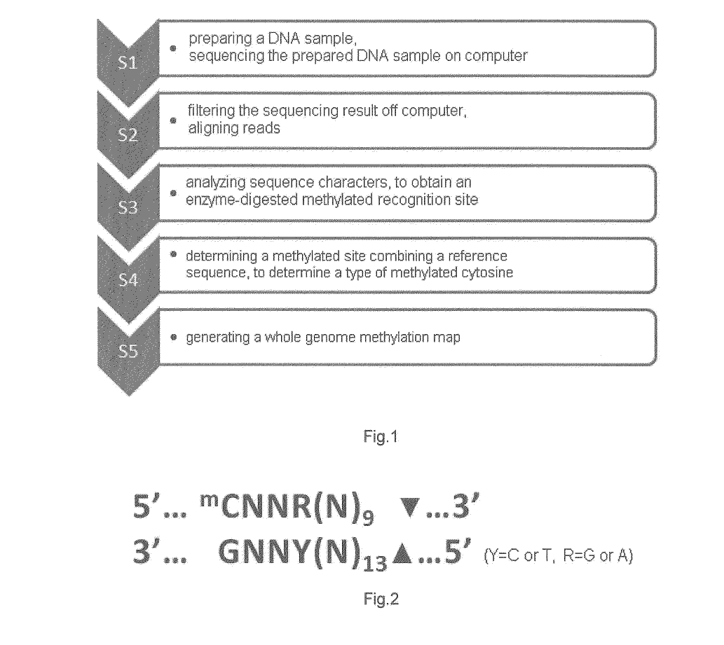

As shown in FIG. 2, MspJI recognizes a methylated double-stand site in the context of CNNR(R=A or G), of which a complementary strand is YNNG(Y=T or C), and introduces double-stranded breaks at fixed distances of 9 bp and 13 by on R end, leaving a four-base 5′ overhang. If a recognition site is fully methylated, then an enzyme-digested fragment having a length of 32 bases or 31 bases is yielded by two-way cleavage. By then, the methylated site is contained in the middle of the enzyme-digested fragment, by which can be enriched for sequencing analysis and alignment, i.e., a position of methylated cytosine in a genome may be known. Since most methylations occur in a form of being fully methylated in sequence CpG, CHG or CHH, while these sequences are mainly recognized and cut by MspJI to yield fragments having a length of 30 by to 32 bp, considering a diversity of recognition site types and a 1 by to 2 by fluctuation of the breaking site, enzyme-digested fragments having a length of 28 by to 34 by are taken as an example for sequencing analysis and alignment, to obtain sequence information comprising these methylated sits.

FIG. 1 is a realization process of detecting a DNA methylation of the present disclosure, which is specifically described below.

In step S1, although any commonly-used sequencing technology in the art may be used for sequencing, as the enzyme-digested fragments are relative short sequences, SE50 is preferred for sequencing. Other high-throughput sequencing technology may also be used in the present disclosure, for example, Illumina GA sequencing technology, or other existing high-throughput sequencing technology.

In step S2, the sequencing result off computer is preferably subjected to a filtration to remove an unqualified read. For example, the unqualified read comprises following two cases: more than 50% bases having a sequencing quality below a certain threshold in all bases of a read; and more than 10% uncertain bases (such as N in Illumina GA sequencing result) in all bases of a read. A low-quality threshold may be determined by those skilled in the art according to specific sequencing technology and sequencing environment. After the unqualified read has been removed, the qualified read is preferably subjected to screening, to retain an intact read without a sequencing adaptor and a read having a length of 28 by to 34 by after trimming off the sequencing adaptor.

The filtered and/or screened reads are preferably aligned to a genome sequence of a species to which the DNA sample belongs, to realize a whole genome location of a read, i.e., an enzyme-digested fragment. Considering the read is generally relative short, a case of being unable to be located by none alignments or multiple alignments may occur, an alignment software is preferably used, for example Soap2.20 is used for twice alignments: 1) by setting a software parameter, the read is aligned to the reference sequence with 2 allowed mismatches in each seed sequence and maximal 4 mismatches in each of the reads, to obtain a first aligned result; 2) by resetting Soap2.20 parameter, the read aligned to multiple positions and an unaligned read in the first aligned result are aligned to the reference sequence without allowed mismatches, to obtain a second aligned result; 3) the first aligned result and the second aligned result are merged together, for calculating an aligning rate and a unique aligning rate. Other short sequences may also be used in a mapping program to realize the alignment.

In step S3, a position of a methylated cytosine on the unique aligning read may be determined in accordance with a relationship between a type and a length of the enzyme-recognized site, and be categorized according to a feature of the read which the methylated cytosine locates. Firstly, whether a methylated cytosine exists in a unique aligning read is determined according to MspJI enzyme digestion features, if a corresponding MspJI recognition site is found at a digested end within a certain distance, then a cytosine in the corresponding MspJI recognition site is a methylated cytosine. Considering a fluctuation of 1 base to 2 bases at the digested site, the enzyme-digested fragments having a length of 28 by to 34 by are classified into 8 types of fragments containing fully methylated recognition site (corresponding C and G site in a complementary strand are all methylated sites): YNCGNR, YCNGR, CNNG, GNNC, CYNRG, CNYRNG, YNNGCNNR and YNNGNCNNR, as well as 2 types of fragments containing a semi-methylated recognition site: CNNR and YNNG, totally 10 types, each type of fragments corresponds to one type of fragment length. It should note that, when being subjected to calculation combining the enzyme-digested site and the type of the read which the methylated cytosine locates, two types of CHG and CHH are unable to be accurately categorized, an overlapping exists between the types (for example, TCCGGA fragment may be any one in two types of YNCGNR or YCNGR), even so, such classification still proved a great convenience for searching and locating a methylated cytosine site based on a relationship between a fragment length and a type of recognition site.

In step S4, a position of a methylated cytosine in a genome is located according to the type of recognition site in each read, combining with an aligning position in Arabidopsis reference genome (TAIR8), and then a basic type of such methylated cytosine is finally determined (i.e., CG, CHG or CHH). Distributions of every recognition site and cytosine type are calculated, the feature of each sequence type is described using SeqLogo.

In step 5, after the methylated cytosine is determined and classified, a sequencing depth of each determined methylated cytosine site is calculated, to yield a file similar to methylated single nucleotide annotation in BS sequencing, for detailed describing information such as chromosome in which each methylated cytosine site locates, sequence coordinate, forward or reverse strand, coverage depth, enzyme-digested recognition site, cytosine type, which are subjected to a calculation to finally determine a total volume and a coverage status of the determined methylated cytosine site, so as to provide status of whole genome MspJI-digested methylation. An exemplary file layout similar to methylated single nucleotide annotation in BS sequencing is specifically shown below:

| | | | | | | |
|---|---|---|---|---|---|---|
| Chr1 | 17 | + | 3 | CNNR | CTAA | CHH |
| Chr1 | 24 | + | 3 | CNNR | CTAA | CHB |
| Chr1 | 1649 | + | 8 | YNCGNR | TACGAA | CG |
| Chr1 | 1650 | − | 10 | YNCGNR | TACGAA | CG |

the first array: chromosome number;
the second array: position of cytosine site;
the third array: information of forward or reverse strand;
the fourth array: the number of reads covered by methylation;
the fifth array: type of recognition site;
the sixth array: specific site sequence;
the seventh array: type of C site;

In the present disclosure, other relative analysis may also be performed, i.e., combining characteristic of the used plant genome, a distribution of methylated cytosine in the genome is also analyzed, for example, a distribution in each element of gene, a distribution in a repetitive sequence region and a distribution of some local regions, etc.

### EXAMPLE

Sample: one whole genome sample of Columbia Arabidopsis leaves;

Sequencing strategy: single ends (SE) Illumina sequencing datasets ;

Specific operational procedure was illustrated below combining with FIG. 1.

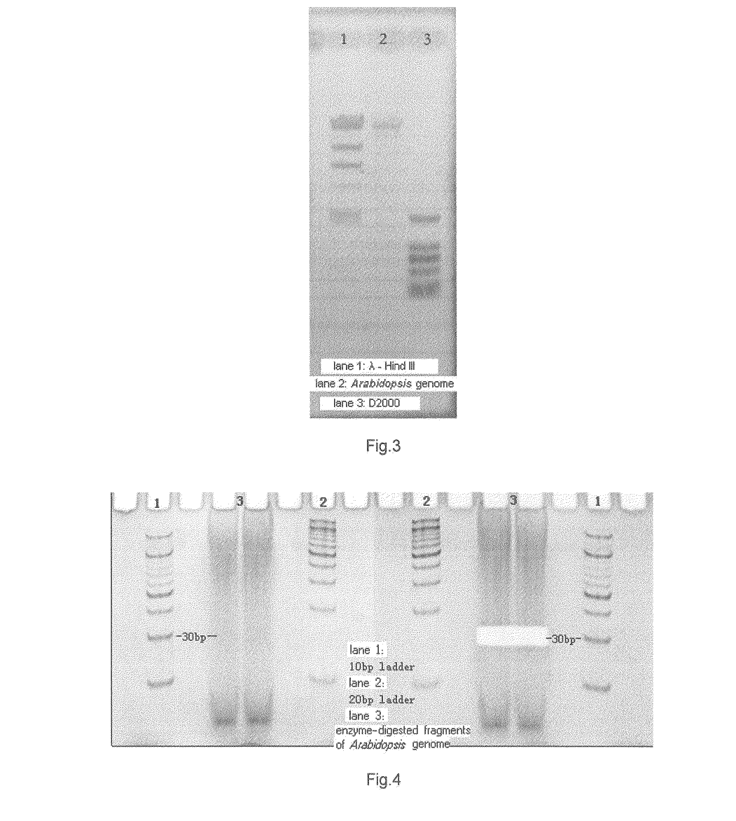

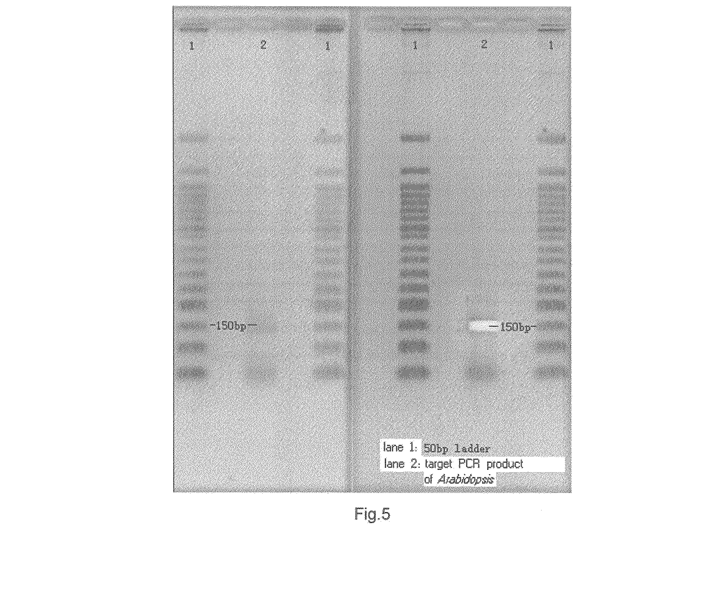

Step S1 comprised several steps: DNA extraction, enzyme digestion, selection and recycling of enzyme-digested fragments, SE library construction, sequencing on computer. Genome DNA was extracted from the Arabidopsis leaves using cetyltrimethylammonium bromide (CTAB) method followed by phenol: chloroform extraction and ethanol precipitation. The genome DNA sample, after checked by 1% agarose gel electrophoresis to obtain those qualified (FIG. 3), were subjected to enzyme digestion using MspJI (purchased from New England Biolabs (NEB)). On the basis of a recommending enzyme digestion system which NEB website provided for MspJI product, following improvements were made directing to a plant genome: 1.5 μg of Arabidopsis genome DNA was enzyme-digested using 12 U (3 μL) MspJI enzyme, in the presence of 0.8 μM oligonucleotides activator, to significantly improve original enzyme digestion effect. After 16 hours, the enzyme-digested DNA was subjected to a 15% native polyacrylamide gel, electrophoresis, and a narrow-band containing those enzyme-digested fragments around 26 by to 38 by was excised in reference of 10 by DNA ladder (FIG. 4). The excised DNA was isolated by Crush and Soak Method and purified by ethanol precipitation, the purified short fragments were used to construct DNA library. Ranges of the recycled fragments were enlarged, with a purpose of detecting a methylated cytosine mostly existed as a non-CpG form in Arabidopsis genome. The library-constructing method referred to the Illmina Pair-End protocol including procedures of DNA end-repair, ‘A’ BASE addition, adaptor ligation and PCR amplification, and the obtained products having a length of 146 by to 158 bp, in which phenol: chloroform extraction and ethanol precipitation were used to purify the products of each process. The PCR products were checked and recycled by 2% agarose gel electrophoresis (FIG. 5), purified according to QIAquick gel extraction kit, and the obtained library was analyzed by Bioanalyzer analysis system before subjected to SE50 sequencing with Illumina HiSeq2000 sequencer.

In step S2, the sequencing result off computer was preferably subjected to a filtration to remove an unqualified read, comprising following two cases: more than 50% bases having a sequencing quality below a certain threshold in all bases of a read; and more than 10% uncertain bases (such as N in Illumina GA sequencing result) in all bases of a read. After the unqualified read had been removed, the qualified read was preferably subjected to screening, to retain an intact read without a sequencing adaptor and a read having a length of 28 by to 34 by after trimming off the sequencing adaptor.

The filtered and/or screened reads were preferably aligned to a genome sequence of a species to which the DNA sample belonged, to realize a whole genome location of a read, i.e., an enzyme-digested fragment. Considering the read is generally relative short, a case of being unable to locate by none alignments or multiple alignments would occur, an alignment software Soap2.20 (obtained from soap.genomics.org.cn/) was used for twice alignments: 1) by setting a software parameter, the read was aligned to the reference sequence with 2 allowed mismatches in each seed sequence and maximally 4 mismatches in each of the reads, to obtain a first aligned result; 2) by resetting Soap2.20 parameter, the read aligned to multiple positions and an unaligned read in the first aligned result were aligned to the reference sequence without allowed mismatches, to obtain a second aligned result; 3) the first aligned result and the second aligned result were merged together, for calculating an aligning rate and a unique aligning rate, referring to Table 1. The table 1 showed specific data volume off computer, obtained data volume after filtration and screening, and the total number of sequence unique aligning to Arabidopsis genome after alignments in the Arabidopsis sample. As the enzyme-digested sequence was relative short and an actual distribution of the methylated site, the unique aligning rate was relative low.

TABLE 1
Statistics of data output, filtration and alignment of Arabidopsis

| | | | | uniquely |
|---|---|---|---|---|
| | original | filtered | aligned | aligned |
| Sample | reads | reads | reads | reads |
| Arabidopsis | 43578097 | 32107319 | 26222436 | 6002281 |
| | | (100%) | (81.67%) | (18.69%) |

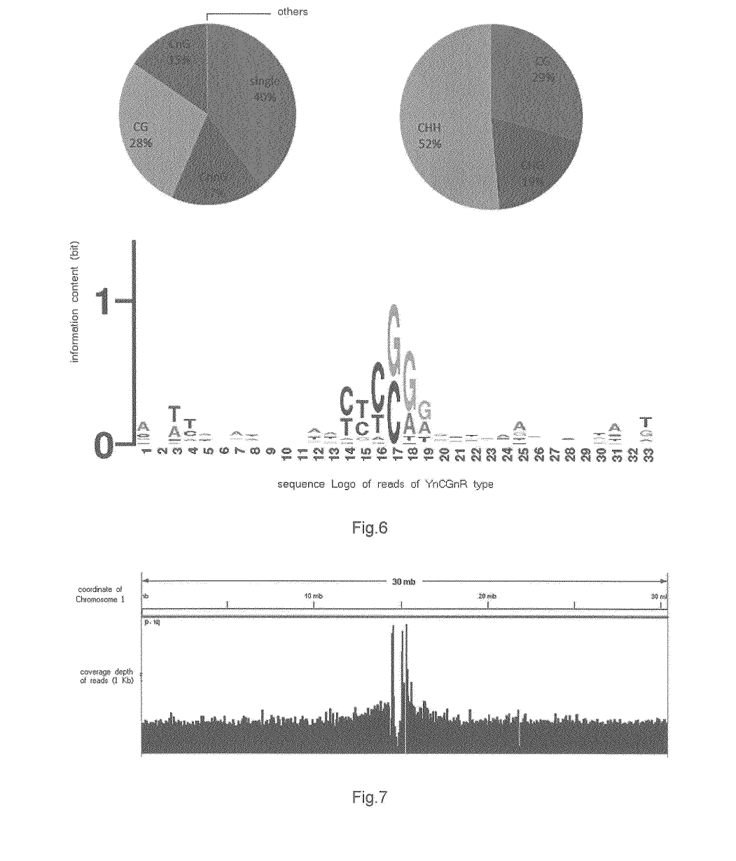

In step S3, a position of a methylated cytosine on the unique aligning read would be determined in accordance with a relationship between a type and a length of the enzyme-recognized site, and be categorized according to a feature of the read which the methylated cytosine locates. Firstly, whether a methylated cytosine exists in a unique aligning read was determined according to MspJI enzyme digestion features (FIG. 6), if a corresponding MspJI recognition site was found at a digested end within a certain distance, then a cytosine in the corresponding MspJI recognition site was a methylated cytosine. Considering a fluctuation of 1 base to 2 bases at the digested site, the enzyme-digested fragments having a length of 28 by to 34 by were classified into 8 types of fragments containing fully methylated recognition site (corresponding C and G site in a complementary strand are all methylated sites): YNCGNR, YCNGR, CNNG, GNNC, CYNRG, CNYRNG, YNNGCNNR and YNNGNCNNR, as well as 2 types of fragments containing a semi-methylated recognition site: CNNR and YNNG, totally 10 types, referring to Table 2 and Table 3. The table 2 showed distributions of coverage and depth with reads which were determined containing the methylated cytosine in every chromosome. The table 3 showed statistical types of the uniquely aligned reads containing the methylated cytosine recognition site, it should note that, the meaning of such classification was to provide convenience for searching and locating the methylated cytosine site based on a relationship between a fragment length and a type of recognition site, however a repetitive statistics existed among different types of site during calculating reads (for example TCCGGA fragment would be calculated twice respectively by two types of YNCGNR and YCNGR. But it still could be seen that, site types of YNCGNR and YCNGR, as well as a one-way enzyme-digested site occupied a relative large proportion in all types.

TABLE 2
Statistical distributions of coverage and depth
with reads which were determined to contain the
methylated cytosine in every chromosome.

| | | total | coverage | |
|---|---|---|---|---|
| | | length | length | depth |
| chromosome | reads | (bp) | (bp) | (X) |
| Chr1 | 858094 | 27588022 | 8430027 | 3.27 |
| Chr2 | 809360 | 25849788 | 5822740 | 4.44 |
| Chr3 | 1224824 | 39586855 | 6872663 | 5.76 |
| Chr4 | 923907 | 30126662 | 5460987 | 5.52 |
| Chr5 | 1278842 | 41120637 | 7777612 | 5.29 |
| ChrC | 882831 | 28667142 | 246026 | 116.52 |
| Total | 5977858 | 192939106 | 34610055 | 5.57 |

TABLE 3
Statistical types of the uniquely
aligned reads containing the
methylated cytosine recognition site

| | | |
|---|---|---|
| YNCGNR | 920954 | 15.34% |
| YCNGR | 418696 | 6.98% |
| YNNGCNNR | 183789 | 3.06% |
| YNNGNCNNR | 193914 | 3.23% |
| CLANG | 449739 | 7.49% |
| GNNC | 226264 | 3.77% |
| CYNRG | 3438 | 0.06% |
| CNYRNG | 2191 | 0.04% |
| CNNR | 863932 | 14.39% |
| YNNG | 713926 | 11.89% |
| NA | 2025438 | 33.74% |
| Total | 6002281 | 100.00% |

In step S4, a position of a methylated cytosine in a genome was located according to the type of recognition site in each read, combining with an aligning position in Arabidopsis reference genome (TAIR8), and then a basic type of such methylated cytosine was finally determined (i.e., CG, CHG or CHH). Distributions of every recognition site and cytosine type were calculated, the feature of each sequence type is described using SeqLogo, referring to FIG. 7. FIG. 7 showed a distribution trend of a methylated cytosine site which was determined in Chromosome 1 of Arabidopsis, a general distribution trend could be seen from FIG. 7: the methylated cytosine sites intensively distributed around a centromere.

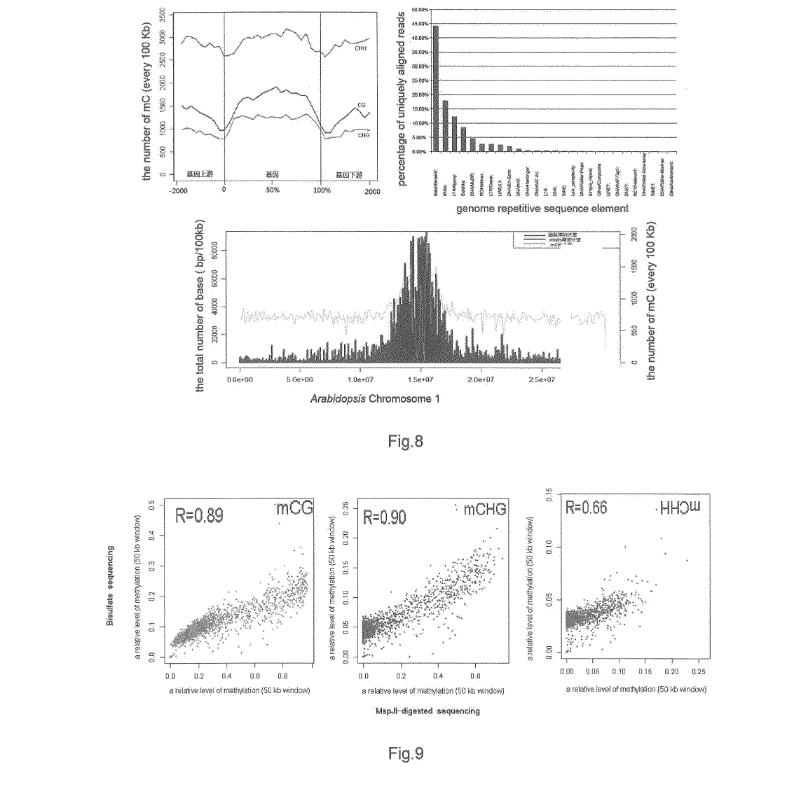

In step 5, after the methylated cytosine is determined and classified, a sequencing depth of each determined methylated cytosine site is calculated, to yield a file similar to methylated single nucleotide annotation in BS sequencing, for detailed describing information such as chromosome in which each methylated cytosine site locates, sequence coordinate, forward or reverse strand, coverage depth, enzyme-digested recognition site, cytosine type, which are subjected to a calculation to finally determine a total volume and a coverage status of the determined methylated cytosine site, so as to provide status of whole genome MspJI-digested methylation, referring to FIG. 8.

The upper left panel in FIG. 8 showed all Arabidopsis genes and a distribution of every captured methylated cytosine within a range of 2000 by upstream and downstream thereof The entire distribution was in consistence with previous discoveries, i.e., the gene region had a heavier methylated level in relative to upstream and downstream thereof, the relative level of methylation around TSS site is very low; the upper right panel in FIG. 8 showed a distribution of all enzyme-digested fragments in the repetitive sequence elements, with approximately 45% fragments located in the repetitive sequence elements; the bottom panel in FIG. 8 also showed distributions of the number of the methylated cytosine in Arabidopsis chromosome 1, coverage length of read and length of repetitive sequence, from which a relationship between a distribution of methylated cytosine and a repetitive sequence could be seen.

An exemplary file layout similar to methylated single nucleotide annotation in BS sequencing is specifically shown below:

| | | | | | | |
|---|---|---|---|---|---|---|
| Chr1 | 17 | + | 3 | CNNR | CTAA | CHH |
| Chr1 | 24 | + | 3 | CNNR | CTAA | CHB |
| Chr1 | 1649 | + | 8 | YNCGNR | TACGAA | CG |
| Chr1 | 1650 | − | 10 | YNCGNR | TACGAA | CG |

the first array: chromosome number;
the second array: position of cytosine site;
the third array: information of forward or reverse strand;
the fourth array: the number of reads covered by methylation;
the fifth array: type of recognition site;
the sixth array: specific site sequence;
the seventh array: type of C site;
and other relative analysis were also performed, i.e., combining characteristic of the used plant genome, a distribution of methylated cytosine in the genome as also analyzed, for example, a distribution in each element of gene, a distribution in a repetitive sequence region and a distribution of some local regions, etc, referring to FIG. 9. FIG. 9 showed a correlation between mCG, mCHG, mCHH sites and BS sequencing data (experimental steps were shown below) in a corresponding region. An X-coordinate in FIG. 9 was a methylated level obtained by this enzyme-digested sequencing, a Y-coordinate in FIG. 9 was a methylated level obtained by BS sequencing, a length of a designated region was 50 Kb, it could be seen from the correlation in FIG. 9, mCG and mCHG had a higher correlation in relative to mCHH. Such result was in consistence with the already known in the art, which indicated the effectiveness of the method in the present disclosure.

Following experimental steps were performed using the genome DNA sample same as the above described, to obtain BS sequencing data.
1. Genome DNA was extracted from the Arabidopsis leaves using cetyltrimethylammonium bromide (CTAB) method followed by phenol: chloroform extraction and ethanol precipitation. The genome DNA sample, after checked by 1% agarose gel electrophoresis to obtain those qualified was fragmented by ultrasonic method to obtain fragments having a length of 100 by to 300 bp.
The library-constructing method referred to the Illmina Pair-End protocol including procedures of DNA end-repair, ‘A’ BASE addition, adaptor ligation and PCR amplification. Phenol: chloroform extraction and ethanol precipitation were used to purify the products of each process.
2. In accordance to specification provided by manufacturer, the obtained genome DNA sample was subjected to bisulfite treatment using ZYMO EZ DNA Methylation-Gold kit (commercially obtained from http://www.bioon.com.cn/reagent/showproduct.asp?id=6078).
3. DNA obtained in step 2 was checked and recycled by 2% agarose gel electrophoresis, purified according to QIAquick gel extraction kit, subjected to a size-selection of library, and PCR amplification. Then the amplified DNA was subjected to a size-selection of library again, the obtained library was analyzed by Bioanalyzer analysis system before subjected to SE50 sequencing with Illumina HiSeq2000 sequencer.

The above descriptions are just general examples of the present disclosure, which are not constructed to limit the present disclosure, and any amendments, equivalent replacements or improvements, etc can be made in the embodiments without departing from spirit, principles and scope of the present disclosure.

1. A method of detecting a genome DNA methylation, comprising following steps:
1) digesting a genome DNA sample with MspJI, to obtain fragments,
2) sequencing the fragments, to obtain reads;
3) aligning the reads to a reference sequence, to select a uniquely aligned read; and
4) determining a site in the reference sequence being methylated in the uniquely aligned read;
wherein the site in the reference sequence corresponds to a C site in at least one of YNCGNR, YCNGR, CNNG, GNNC, CYNRG, CNYRNG, YNNGCNNR, YNNGNCNNR, CNNR, YNNG and a complementary strand thereof, wherein Y is C or T, R is A or G, N is A, C, T or G, and H is C, A or T.

2. The method of claim 1, wherein the step 1) further comprises:
enriching the fragments having a length of 28 by to 34 by after the digesting.

3. The method of claim 1, wherein in the step 2), the sequencing is performed on illumina solexa, ABI SOLID and/or Roche 454 sequencing platform.

4. The method of claim 1, wherein the step 3) further comprises:
3-1) aligning the reads to the reference sequence with 2 allowed mismatches in each seed sequence and maximal 4 mismatches in each of the reads, to obtain a first aligned result;
3-2) aligning reads aligned to multiple positions and unaligned reads in the step 3-1) to the reference sequence without allowed mismatches, to obtain a second aligned result; and
3-3) merging the first aligned result and the second aligned result.

5. A method of analyzing a genome methylation, comprising following steps:
1) digesting a genome DNA sample with MspJI, to obtain fragments,
2) sequencing the fragments, to obtain reads;
3) aligning the reads to a reference sequence, to select a uniquely aligned read;
4) determining a methylated C site in the uniquely aligned read, to determine a corresponding site in the reference sequence being methylated,
wherein the methylated C site is a C site in at least one of YNCGNR, YCNGR, CNNG, GNNC, CYNRG, CNYRNG, YNNGCNNR, YNNGNCNNR, CNNR, YNNG and a complementary sequence thereof, wherein Y is C or T, R is A or G, N is A, C, T or G, and H is C, A or T;
5) calculating a type distribution of CG, CHG or CHH in the methylated C site, wherein H is C, A or T;
6) annotating following one or more kinds of information in a whole genome map, to obtain a whole genome methylation map, comprising:
a sequencing depth of each methylated C site;
information, comprising methylated single nucleotide annotation, No. of chromosome in which each determined methylated C site locates, a C site position, forward or reverse strand, a coverage depth, a digested and recognized site, types of cytosine; and a total amount and coverage of the methylated cytosine position.

6. The method of claim 5, wherein the step 1) further comprises:
enriching the fragments having a length of 28 by to 34 by after the digesting.

7. The method of claim 5, wherein in the step 2), the sequencing is performed on illumina solexa, ABI SOLiD and/or Roche 454 sequencing platform.

8. The method of claim 5, wherein the step 3) further comprises:
3-1) aligning the reads to the reference sequence with 2 allowed mismatches in each seed sequence and maximal 4 mismatches in each of the reads, to obtain a first aligned result;
3-2) aligning reads aligned to multiple positions and unaligned reads in the step 3-1) to the reference sequence without allowed mismatches, to obtain a second aligned result; and
3-3) merging the first aligned result and the second aligned result.

9. The method of claim 5, wherein the MspJI is a modification-dependent restriction enzyme.

10. The method of claim 1, wherein the MspJI is a modification-dependent restriction enzyme.
